(19) United States
(12) Patent Application Publication
(10) Pub. No.: US 2002/0049416 A1
(43) Pub. Date: Apr. 25, 2002

(54) Disposable bag for storing excrement and solid and liquid residues for use in hospital containers

(76) Inventor: Marcos Aparecido Savoia, Sao Paulo (BR)

Correspondence Address: ADAMS & WILKS, 31st Floor, 50 Broadway, New York, NY, 10004, US

(21) Appl. No.: 09/838,056

(22) Filed: Apr. 19, 2001

(30) Foreign Application Priority Data
Apr. 19, 2000 (BR) PI 0001235-1

(51) Int. Cl. A61M 1/00

(52) U.S. Cl. 604/327

(57) ABSTRACT

A disposable bag for excrement and solid and liquid residues has a tubular body made of a flexible material and having an open end, a closed end, an interior space for receiving excrement and solid and liquid residues through the open end, and a peripheral edge at the open end having an internal channel. A polymeric material is disposed in the interior space of the tubular body for solidifying liquid residues in contact therewith. At least one strip of material is disposed in the internal channel of the tubular body for drawing the tubular body into a generally pouch shape and for sealing the tubular body.

### BACKGROUND OF THE INVENTION

1. Field of the Invention

The present invention relates generally to disposable bags and, more specifically, to a disposable bag for storing excrement (urine and feces) or other solid and liquid residues for use in hospital containers, such as bedpans, basins, buckets, sinks and the like, with the specific purpose of avoiding the direct contact between the patient and the receiving container of the excrement or solid and liquid residues in general in order to avoid the occurrence or at least substantially minimize the occurrence of hospital infections caused by such means of transmission.

2. Background of the Invention

In hospitals, clinics and the like, many patients require the assistance of third parties, such as nurses, hospital employees or a companion, when desiring to relieve nature via bowel movements. Nurses and hospital employees are theoretically trained and thus capable of preventing patients from using, prior to cleaning and disinfecting, containers for storing excrement and solid and liquid residues in general which have been used by other patients. However, even in situations where nurses or hospital employees have been carefully trained, there is always the risk that a bedpan or basin which has been removed from a patient is inadvertently used by another patient prior to being properly cleaned and disinfected. Furthermore, it is undeniable that when using a container such as a bedpan, basin or the like the patient almost always maintains direct contact with the surface of the container, thereby further increasing the risk of infections.

Thus, after each use, it is necessary to empty and wash bedpans and basins, and sterilization is necessary if they are to be successively used by different patients. Not only are these operations time-consuming and expensive since specially trained hospital personnel and equipment have to be employed, but there additionally exists the danger of germs spreading from the time the bedpans and basins are used until they are emptied and sterilized.

While local authorities and hospital officials have developed and launched campaigns aimed at avoiding hospital infections, the risk of infection in connection with hospital containers for receiving urine, excrement and residues in general has not decreased.

### SUMMARY OF THE INVENTION

It is an object of the present invention to provide a disposable bag which can be readily used with hospital containers for storing excrement and solid and liquid residues to avoid infections through multiple use of the containers by different patients.

Another object of the present invention is to provide a disposable bag which reliably confines excrement and solid and liquid residues therein for considerable lengths of time without leakage.

Another object of the present invention is to provide a disposable bag for storing excrement and solid and liquid residues which can be easily handled without spillage of its content and which can be easily disposed of after use without danger of fouling sanitary equipment or other disposal apparatus.

Another object of the present invention is to provide a disposable bag for storing excrement and solid and liquid residues which can be easily handled and which generally simplifies the work of hospital attendants or the like concerned with the toilet habits of patients.

Another object of the present invention is to provide a disposable bag for storing excrement and solid and liquid residues which simplifies the work of hospital attendants or the like concerned with the toilet habits of patients by providing a convenient and greatly improved sanitary means for collecting and disposing of excrement and solid and liquid residues in general with minimum of malodor, and wherein the container itself thereafter requires very little cleaning and sanitizing in contrast to ordinary practices.

Another object of the present invention is to provide a disposable bag for storing excrement and solid and liquid residues which provides comfort and dignity to the user.

Another object of the present invention is to provide a disposable bag for storing excrement and solid and liquid residues which avoids air contamination after use.

Another object of the present invention is to provide a disposable bag for storing excrement and solid and liquid residues which can be mass-produced economically, is easy to use, can be easily handled, and generally simplifies the work of hospital attendants or the like concerned with the toilet habits of patients.

Still a further object of the present invention is to provide a disposable bag for storing excrement and solid and liquid residues which prevents access to vectors (e.g., flies) into the interior of the disposable bag after use.

The foregoing and other objects of the present invention are carried out by a disposable bag for storing excrement and solid and liquid residues for use in hospital containers, such as bedpans, basins, buckets and sinks. The disposable bag comprises a tubular body made of a flexible material and having an open end, a closed end, an interior space for receiving excrement and solid and liquid residues through the open end, and a peripheral edge at the open end having an internal channel. A polymeric material is disposed in the interior space of the tubular body for solidifying liquid residues in contact therewith. A plurality of strips of material are disposed in the internal channel of the tubular body for drawing the tubular body into a pouch shape and sealing it after use.

The polymeric material preferably comprises a gel substance, such as sodium polyacrilate. The tubular body is preferably made of low density polyethylene.

### BRIEF DESCRIPTION OF THE DRAWINGS

The foregoing summary, as well as the following detailed description of a preferred embodiment of the invention, will be better understood when read in conjunction with the accompanying drawings. For the purpose of illustrating the invention, there is shown in the drawings an embodiment which is presently preferred. It should be understood, however, that the invention is not limited to the precise arrangement and instrumentalities shown. In the drawings:

FIG. 1 is a side view of a disposable bag according to an embodiment of the present invention with a gel for solidifying liquid residues represented in dashed lines;

FIG. 2 shows a step in the process of placing the disposable bag of FIG. 1 in a bedpan;

FIG. 3 shows a step in the process of removing the disposable bag of FIG. 1 from the bedpan;

FIG. 4 is a perspective view showing the disposable bag in a closed state during the process of removing the disposable bag from the bedpan;

FIG. 5 is a perspective view showing the application of the disposable bag of FIG. 1 to a basin;

FIG. 6 is a perspective view showing the application of the disposable bag of FIG. 1 to a bucket;

FIG. 7 is a perspective view showing the application of the disposable bag of FIG. 1 to a generally rectangular container;

FIG. 8 is a perspective view showing the application of the disposable bag of FIG. 1 to a container coupled to a chair; and

FIG. 9 shows the disposable bag of FIG. 1 in a sealed state after use.

### DETAILED DESCRIPTION OF THE PREFERRED EMBODIMENT

While this invention is susceptible of embodiments in many different forms, this specification and the accompanying drawings disclose only one form as an example of the use of the invention. The invention is not intended to be limited to the embodiment so described, and the scope of the invention will be pointed out in the appended claims.

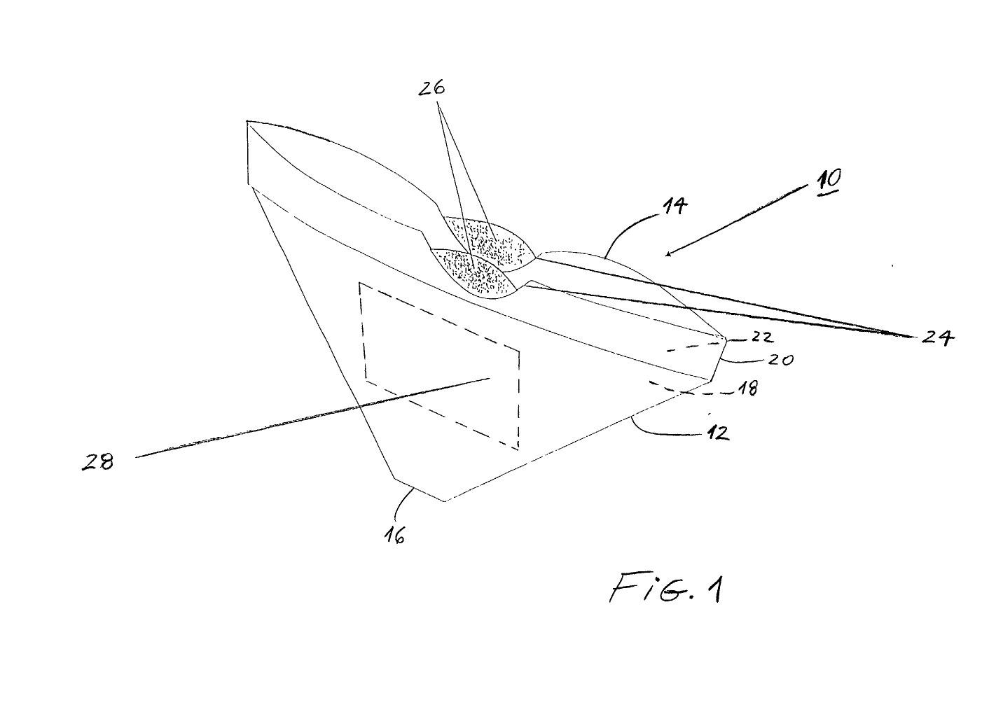

Referring now to the drawings in detail, wherein like numerals are used to indicate like elements throughout, there is shown in FIGS. 1-9 an embodiment of a disposable bag, generally designated at 10, according to the present invention for storing excrement and solid and liquid residues for use in hospital containers, such as bedpans, basins, buckets, sinks and the like. The specific purpose of the disposable bag 10 is to prevent direct contact between the user and the receiving container of excrement and solid and liquid residues in general in order to avoid the occurrence or at least substantially minimize the occurrence of infections caused by such means of transmission.

Referring to FIG. 1, the disposable bag 10 is shown in a partly closed state and comprises a tubular body 12 made of a collapsible flexible material and having an open end 14, a closed end 16, an interior space 18 for receiving excrement and solid and liquid residues through the open end 14, a peripheral edge 20 at the open end 14 having an internal channel 22, and two generally central openings 24. The internal channel 22 is formed, for example, by folding the edge of the tubular body 12 at the open end 14 and then sealing (e.g., using an adhesive or by heat sealing) the edge to an adjacent portion of the tubular body to form a channel structure. In an alternative embodiment, the internal channel 22 may be formed using a separate piece of collapsible flexible material and then connected to the tubular body 12 by suitable sealing means.

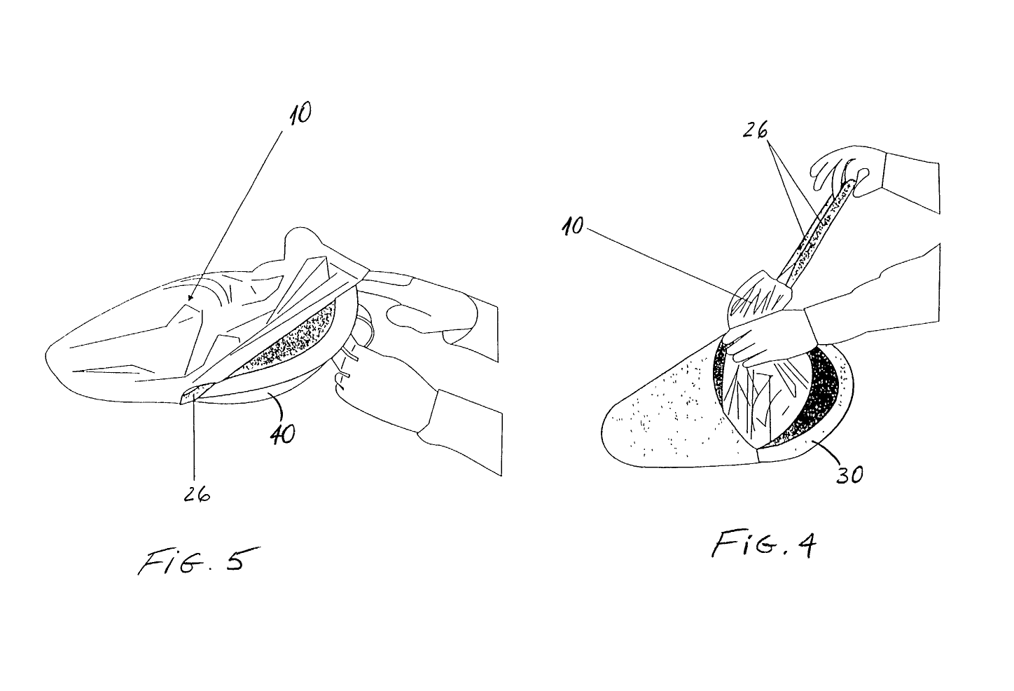

Strips of material 26 are disposed in the internal channel 22 of the tubular body 12 and are accessible through the openings 24 for drawing the tubular body 12 into a pouch shape and sealing it after use, as shown in FIGS. 4 and 9. In an alternative embodiment, the strips of material 26 may be attached to the peripheral edge 20 by adhesive, stitching or other means instead of the internal channel 22.

A polymeric material 28 is disposed in the interior space 18 of the tubular body 12 for solidifying liquid residues in contact with the polymeric material 28. The polymeric material 28 preferably comprises a gel substance, such as sodium polyacrilate, which is impregnated between two layers of non-woven material. The layers containing the gel substance is attached to an interior wall portion of the tubular body 12 using a suitable adhesive or heat bonding process. When the polymeric material 28 is brought into contact with a liquid, the polymeric material absorbs the liquid, reacts chemically with the liquid and is transformed into a pasty substance. Thus, the liquid solidifies upon contact with the polymeric material 28 and leakage thereof during use and transportation of the disposable bag is prevented.

The collapsible tubular body 12 preferably comprises a single layer of a flexible material which is formed into the tubular body using any conventionally known process. The flexible material is preferably a suitable plastic material, such as low density polyethylene. In the embodiment shown in FIG. 1, the tubular body 12 is generally trapezoidal-shaped. However, it is understood that the shape of the tubular body 12 may be other than generally trapezoidal, such as generally square or circular, depending upon the configuration of the container with which it will be used.

The strips of material 26 are preferably made of the same flexible material as the tubular body 12 and are fused to the tubular body 12. Alternatively, instead of the strips of material 26, drawstrings may be disposed in the internal channel 22 with portions of the drawstrings arranged to be accessible through the openings 24 of the tubular body 12 for drawing the tubular body 12 into a pouch shape and sealing it after use, as shown in FIGS. 4 and 9.

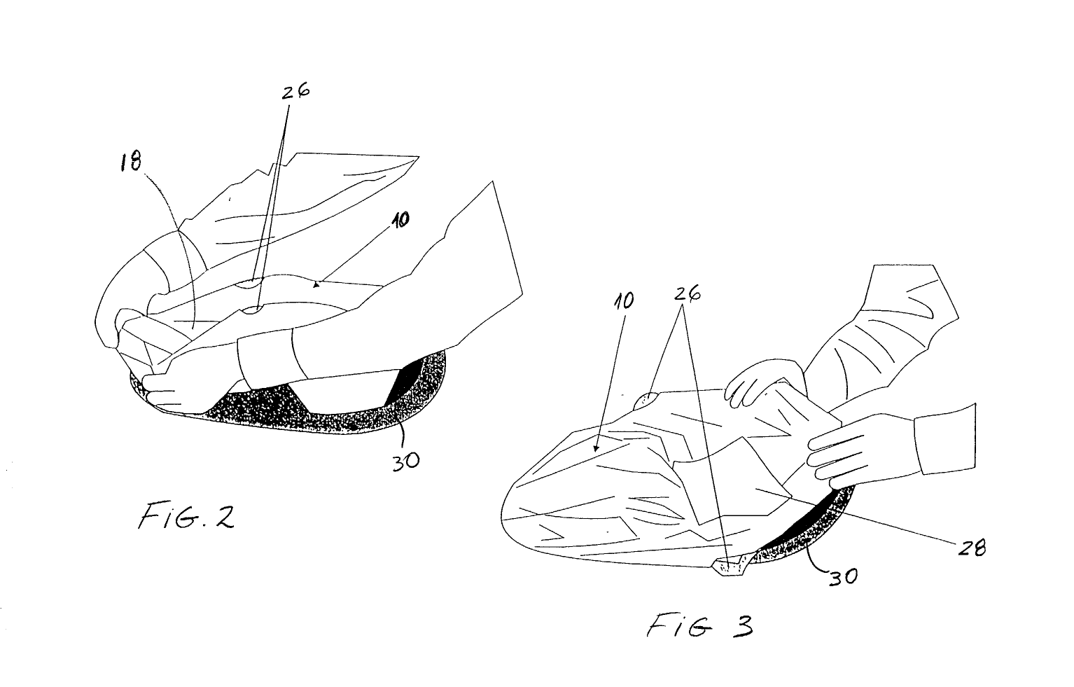

FIG. 2 shows a manner of assembling the disposable bag 10 over a bedpan 30. The collapsible disposable bag 10 is opened and the peripheral edge 20 thereof is disposed over upper front edges of a bedpan 30 so that portions of the bedpan 30 which are likely to be contacted by an individual during use are completely covered by the tubular body 12. The closed end 16 and outer side wall portions of the tubular body 12, including the polymeric material 28, are then inserted into the bedpan 30 to line inner surfaces of the bedpan 30 so that the inside of the bedpan 30 is covered with the tubular body and the interior space 18 of the tubular body 12 is accessible to receive excrement and solid and liquid residues during use. After use, the disposable bag 10 is removed from the bedpan 30 (FIG. 3) and tension is applied to the strips of material 26 to draw the tubular member 12 into a pouch shape (FIG. 4). Thus, after use, the disposable bag 10 can be safely removed from the bedpan 30 and immediately sealed (e.g., by tying of the strips of material 26) by someone without direct contact with the contents of the disposable bag or the user thereof.

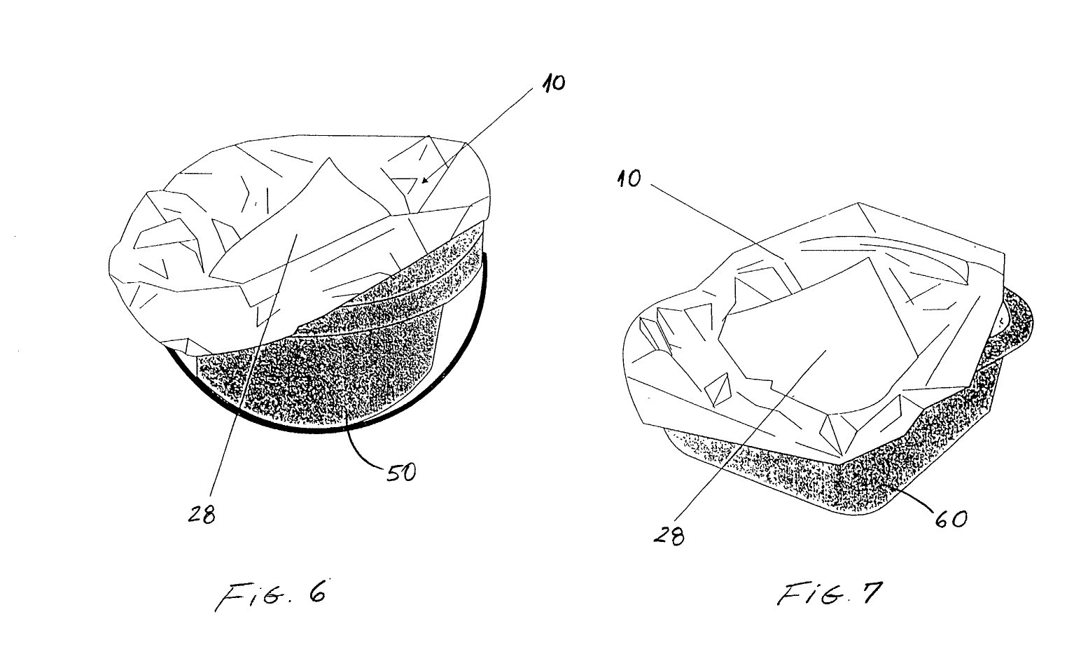

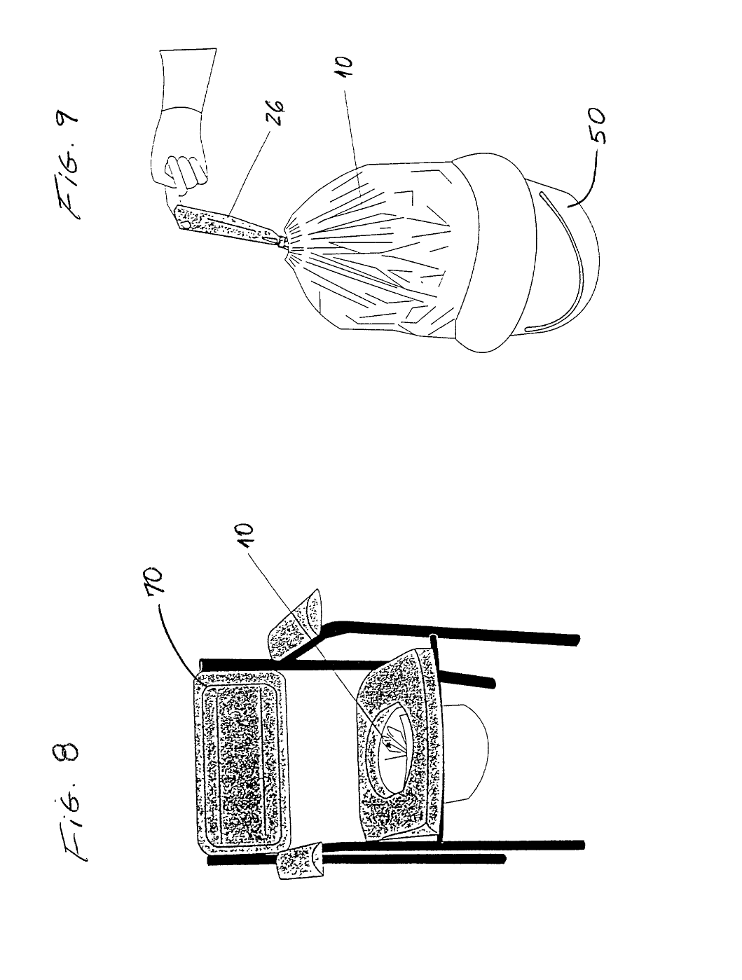

FIGS. 5-8 show the application of the disposable bag 10 to other types of containers, including a basin 40 (FIG. 5), a bucket 50 (FIG. 6), a pan 60 (FIG. 7) and to a container (e.g., a bucket) coupled to a chair 80 (FIG. 8). The manner of installation of the disposable bag 10 to these types of containers is the same as described above for the bedpan 30. FIG. 5 further illustrates a preferred method of removing the disposable bag 10 from the container 40 which includes immobilizing the container 40 with one hand, placing the other hand under the tubular body 12 (i.e., placing the other hand in contact with the exterior surface of the tubular body) with the fingers spaced apart, and then slowly removing the disposable bag with the spaced-apart fingers of the hand.

FIG. 9 shows the disposable bag 10 in a state in which it has been removed from the bucket 50 and in which the strips of material 26 have been tensioned to draw the tubular body 12 into a pouch shape and then tied to seal off odors and conceal from sight the contents. The disposable bag 10, with its contents, may then be carried to a place where it is to be disposed. Furthermore, after the sealed disposable bag 10 has been removed, the container requires only a minimum of cleaning or sanitizing.

By the foregoing construction and use with containers including bedpans, basins, buckets, sinks and the like, the disposable bag according to the present invention prevents direct contact between the user and the receiving container of excrement and solid and liquid residues in general in order to avoid the occurrence or at least substantially minimize the occurrence of infections caused by such means of transmission. Furthermore, by providing the polymeric material in the interior space of the tubular body, any and all liquid which is received in the interior space is solidified. By this construction, the disposable bag can be safely transported without the risk of any liquid leaking from the disposable bag and contaminating the environment and third parties.

Moreover, the disposable bag according to the present invention can be mass-produced economically, is easy to use, is capable of retaining its content without leakage, and can be easily and safely handled. The disposable bag of the present invention generally simplifies the work of hospital attendants or the like concerned with the toilet habits of patients by providing a convenient and greatly improved sanitary means for collecting and disposing of excrement and solid and liquid residues in general with minimum of malodor, and the container itself thereafter requires very little cleaning and sanitizing in contrast to ordinary practices. Furthermore, by its simple construction and manner of use, the disposable bag of the present invention provides comfort and dignity for the patient or user, effectively avoids contamination of the air and surrounding environment, and prevents access to vectors, such as flies, upon disposal of the disposable bag. In certain cases, the disposable bag of the present invention can replace the disposable diaper.

From the foregoing description, it can be seen that the present invention comprises an improved disposable bag for storing excrement and solid and liquid residues in general for use in hospital containers. It will be appreciated by those skilled in the art that the disposable bag may be used with containers outside the hospital, such as at home, the workplace, outdoors and the like, and obvious changes can be made to the embodiment described in the foregoing description without departing from the broad inventive concept thereof. It is understood, therefore, that this invention is not limited to the particular embodiment disclosed, but is intended to cover all obvious modifications thereof which are within the scope and the spirit of the invention as defined by the appended claims.

I claim:

1. A disposable bag for excrement and solid and liquid residues for use with containers, the disposable bag comprising:
a tubular body made of a flexible material and having an open end, a closed end, an interior space for receiving excrement and solid and liquid residues through the open end, and a peripheral edge at the open end having an internal channel; a polymeric material disposed in the interior space of the tubular body for solidifying liquid residues in contact therewith; and at least one strip of material disposed in the internal channel of the tubular body for drawing the tubular body into a generally pouch shape and for sealing the tubular body.

2. A disposable bag according to claim 1; wherein the polymeric material comprises a gel substance.

3. A disposable bag according to claim 2; wherein the gel substance comprises sodium polyacrilate.

4. A disposable bag according to claim 1; wherein the polymeric material comprises sodium polyacrilate.

5. A disposable bag according to claim 1; wherein the tubular body is made of low density polyethylene.

6. A disposable bag according to claim 1; wherein the at least one strip of material comprises two strips of material disposed in the internal channel of the tubular body for drawing the tubular body into a generally pouch shape and for sealing the tubular body.

7. A disposable bag according to claim 6; wherein the tubular body has two openings each providing access to a respective one of the strips of material for drawing the tubular body into a generally pouch shape and for sealing the tubular body.

8. A disposable bag according to claim 1; wherein the tubular body has at least one opening providing access to the strip of material for drawing the tubular body into a generally pouch shape and for sealing the tubular body.

9. A disposable bag according to claim 1; wherein the tubular body is generally trapezoidal-shaped.

10. In combination with a container for containing excrement or other solid and liquid residue, a disposable bag for enveloping the container and for receiving the excrement or other solid and liquid residue, the disposable bag comprising: a flexible tubular body having an open end, a closed end, and an interior space for receiving the excrement or other solid and liquid residue through the open end; means disposed in the interior space of the tubular body for solidifying liquid residues in contact therewith; and means for configuring the tubular body into a generally pouch shape and for sealing the tubular body after receipt of the excrement or other solid and liquid residue.

11. A combination according to claim 10; wherein the means disposed in the interior space comprises a gel substance.

12. A combination according to claim 11; wherein the gel substance comprises sodium polyacrilate.

13. A combination according to claim 10; wherein the tubular body is made of low density polyethylene.

14. A combination according to claim 10; wherein the means for configuring and sealing the tubular body comprises a peripheral edge disposed at the open end of the tubular body having an internal channel and an opening, and at least one strip of material disposed in the internal channel of the tubular body and accessible through the opening of the peripheral edge.

15. A combination according to claim 10; wherein the means for configuring and sealing the tubular body comprises a peripheral edge disposed at the open end of the tubular body having an internal channel and a plurality of openings, and a plurality of strips of material disposed in the internal channel of the tubular body and each accessible through a respective one of the openings of the peripheral edge.

16. A combination according to claim 10; wherein the tubular body is generally trapezoidal-shaped.
